SEQUENCE LISTING (10 sequences)

SEQ ID NO: 1 - DNA, 18 bp, Artificial Sequence
  Forward primer for amplifying portion of PA4943 gene
gaaacggtggcattcgtc

SEQ ID NO: 2 - DNA, 18 bp, Artificial Sequence
  Reverse primer for amplifying portion of PA4943 gene
gtttccagctgggtctcg

SEQ ID NO: 3 - DNA, 20 bp, Artificial Sequence
  Forward primer for amplifying portion of PA3523 gene
ccagcaactgttcctcatcg

SEQ ID NO: 4 - DNA, 18 bp, Artificial Sequence
  Reverse primer for amplifying portion of PA3523 gene
caggtaggtgcgctcgtc

SEQ ID NO: 5 - DNA, 18 bp, Artificial Sequence
  Forward primer for amplifying portion of PA2931 gene
cgaggcgatggaaatcag

SEQ ID NO: 6 - DNA, 20 bp, Artificial Sequence
  Reverse primer for amplifying portion of PA2931 gene
gcatagaaggtcgccaactc

SEQ ID NO: 7 - DNA, 20 bp, Artificial Sequence
  Forward primer for amplifying portion of PA0182 gene
cgacatcctggtcaacaatg

SEQ ID NO: 8 - DNA, 18 bp, Artificial Sequence
  Reverse primer for amplifying portion of PA0182 gene
ggtgatgtaggccgcttc

SEQ ID NO: 9 - DNA, 19 bp, Artificial Sequence
  Forward primer for amplifying portion of PA4167 gene
gcagatctacggcaacgag

SEQ ID NO: 10 - DNA, 20 bp, Artificial Sequence
  Reverse primer for amplifying portion of PA4167 gene
gcaagtaagggctgagttcg

(19) United States
(12) Patent Application Publication
(10) Pub. No.: US 2012/0053155 A1
(43) Pub. Date: Mar. 1, 2012

(54) SYSTEM AND METHOD FOR REVERTING ANTIBIOTIC TOLERANCE OF BACTERIAL PERSISTER CELLS

(75) Inventors: Dacheng Ren, Syracuse, NY (US); Jiachuan Pan, Syracuse, NY (US)

(73) Assignee: Syracuse University, Syracuse, NY (US)

(21) Appl. No.: 13/206,185

(22) Filed: Aug. 9, 2011

Related U.S. Application Data
(60) Provisional application No. 61/371,771, filed on Aug. 9, 2010.

(51) Int. Cl. A61K 31/341; C12N 1/20; A61K 31/65; A61K 31/397; A61K 31/47

(52) U.S. Cl. 514/154; 514/473; 514/210.02; 514/312; 514/210.05; 435/252.1; 435/253.3; 435/252.8; 435/252.2

(57) ABSTRACT

A system and method for reverting the antibiotic tolerance of persister cells. Brominated furanones, which are quorum sensing inhibitors, are used to revert the antibiotic tolerance of persister cells and enhance their susceptibility to antibiotics by up to one hundred fold. Brominated furanones can be used against bacterial persister cells in biofilms or planktonic form.

### CROSS-REFERENCE TO RELATED APPLICATIONS

This application claims priority to U.S. Provisional Patent Application Ser. No. 61/371,771 filed on Aug. 9, 2010 and entitled “System and Method for Waking Persister Cells Using Quorum Sensing Inhibitors,” the entirety of which is hereby incorporated by reference herein.

### BACKGROUND OF THE INVENTION

1. Field of the Invention

The present invention relates to antibiotics and, more particularly, to a system and method for decreasing the tolerance of bacterial persister cells to antibiotics.

2. Description of the Related Art

Bacteria are well known to form metabolically inactive persister cells that are extremely tolerant to almost all antibiotics. The discovery of persister cells dates back to the discovery in 1944 that penicillin could lyse most Staphylococci cells, while a small fraction of the population remained viable even after a prolonged treatment. Increasing evidence has shown that persister cells are not mutants with drug resistance genes, but rather phenotypic variants of the wild-type strain due to unbalanced production of toxins/anti-toxins. Although persister cells normally only make up a small portion of the population (less than 1%), they play a critical role in antibiotic tolerance. Most antibiotics inhibit bacteria by targeting growth-related cellular activities, e.g., protein, DNA, and cell wall syntheses. Antibiotic treatment can eliminate the majority of the bacterial population by killing the normal cells. For persister cells, however, antibiotics can only repress but not kill this subpopulation because persister cells are non-growing dormant cells. Thus, the seeming disadvantage of being dormant in normal environments becomes an advantage for persister cells when being challenged by antibiotics. Persister cells neither die nor grow in the presence of an antibiotic, and when the treatment is stopped, they reestablish the population with a similar percentage of cells as persisters, leading to high-level antibiotic tolerance. Such intrinsic tolerance can lead to chronic infections with recurrence of symptoms and facilitates the development and wide spread of acquired multidrug resistance through genetic mutations. Thus, persister cells are a promising target for developing more effective methods to control chronic infections. However, controlling persister cells is still an unmet challenge.

One approach to eliminating persister cells is to wake up this dormant population and render them to return to a metabolically active stage. These awakened cells will then be sensitive to antibiotics. In Gram-positive bacteria, a 17-kDa protein termed resuscitation-promoting factor (“Rpf”) has been discovered as a potential factor to wake up dormant cells. However, a full wakeup call may cause resumption of bacterial growth, which can lead to adverse progression of infection if the antibiotics are not administered during the right window.

Recently, sugars such as mannitol, glucose, fructose and pyruvate have been shown to generate proton-motive force and promote the uptake of aminoglycosides by persister cells. However, this approach is limited to aminoglycosides and require relatively large amounts of sugar (mM), which may cause complications in vivo.

The absolute number of persister cells in a culture increases significantly when the culture enters stationary-phase and the surface-attached highly hydrated structures known as biofilms. Recent research has demonstrated that quorum sensing (bacterial cell-cell signaling by sensing and responding to cell density) promotes persister formation in Pseudomonas aeruginosa, e.g., acyl-homoserine lactone 3-OC12-HSL and phenazine pyocyanin, both quorum-sensing-related signaling molecules, can significantly increase the persister numbers in logarithmic phase cultures of P. aeruginosa PA01 but not Escherichia coli or Staphylococcus aureus cultures. Consistently, quorum sensing signals and persisters of P. aeruginosa have been isolated from cystic fibrosis patients. These findings bring an opportunity to control persister formation by inferring with these control networks.

### BRIEF SUMMARY OF THE INVENTION

In accordance with the foregoing objects and advantages, the present invention provides a series of quorum sensing inhibitors that can revert the antibiotic tolerance of bacterial persister cells and increase their susceptibility to antibiotics, e.g. ciprofloxacin (“Cip”), by up to 100 times. These compounds can also reduce persister formation at growth non-inhibitory concentrations.

According to one aspect of the invention is a method for reverting the antibiotic tolerance of bacterial persister cells, the method comprising the step of contacting the bacterial persister cell with a brominated furanone. The brominated furanone can be any compound selected from Table 2 or any derivative of the compounds in table 2, including mixtures thereof. The bacterial persister cell can be, for example, Pseudomonas aeruginosa, Burkholderia cepacia, Salmonella typhimurium, Vibrio fisheri, V. harveyi, V. cholera, Aeromonas hydrophila, Serratia liquefaciens, Erwinia carotovora, Agrobacterium tumefaciens, and mixtures thereof, although an enormous variety of other bacterial organisms are possible. The bacterial persister cell can also be in a biofilm, or they can be planktonic. According to one aspect of the invention, application of a brominated furanone does not stimulate full growth of the cells, but does activate a transport activity of the outer membrane of the cell and/or activates transcription of one or more specific genes, usually leading to enhanced susceptibility to antibiotics.

According to a second aspect of the invention is a method to inhibit or eliminate a bacterial infection including bacterial persister cells, the method comprising the steps of: (i) administering a brominated furanone to the bacterial infection, wherein the brominated furanone reverts the antibiotic tolerance of the bacterial persister cell(s); and (ii) administering one or more antimicrobial agents to the bacterial infection. The step of administering a brominated furanone to the bacterial infection comprises contacting the bacterial persister cell(s) with the brominated furanone. The bacterial persister cell can be, for example, Pseudomonas aeruginosa, Burkholderia cepacia, Salmonella typhimurium, Vibrio fisheri, V. harveyi, V. cholera, Aeromonas hydrophila, Serratia liquefaciens, Erwinia carotovora, Agrobacterium tumefaciens, and mixtures thereof, although an enormous variety of other bacterial organisms are possible. The bacterial persister cell can also be in a biofilm, or they can be planktonic. According to one aspect of the invention, application of a brominated furanone does not stimulate full growth of the cells, but does activate a transport activity of the outer membrane of the cell and/or activates transcription of specific genes, usually leading to enhanced susceptibility to antibiotics. According to another aspect of the invention, the antimicrobial agent is selected from the group consisting of a β-lactam, an aminoglacoside, a quinolone, a tetracycline, a cephalosporin, and mixtures thereof. The bacterial infection can be present, for example, in an animal (including a human) or a plant.

According to a third aspect of the invention is a method of preventing a bacterial infection from developing on a surface, the method comprising the steps of: (i) administering a brominated furanone to said surface; and (ii) administering at least one antimicrobial agent to said surface. The step of administering a brominated furanone to the surface comprises contacting the surface with the brominated furanone. The bacterial infection protected against can be, for example, Pseudomonas aeruginosa, Burkholderia cepacia, Salmonella typhimurium, Vibrio fisheri, V. harveyi, V. cholera, Aeromonas hydrophila, Serratia liquefaciens, Erwinia carotovora, Agrobacterium tumefaciens, and mixtures thereof, although an enormous variety of other bacterial infections are possible. According to one aspect of the invention, application of the brominated furanone to the surface causes reversion of—or prevents—antibiotic tolerance of bacterial persister cell(s) that may come in contact with that surface and attempt to establish a bacterial colony and eventual infection, or any persister cells formed after normal bacterial cells are attached. According to another aspect of the invention, the antimicrobial agent is selected from the group consisting of a β-lactam, an aminoglacoside, a quinolone, a tetracycline, a cephalosporin, and mixtures thereof. The surface can be, for example, the surface of an implantable device, a wound dressing, a medical instrument, a cooking or cleaning surface, or any of a wide variety of other surface that preferably remain free of bacterial infection or contamination.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1. is a graph showing that BF8 inhibits quorum sensing based on homoserine lactones;

FIG. 2 is a graph showing BF8 at 100 μg/mL reduced persister formation during 5-h incubation of P. aeruginosa PA01 in LB medium;

FIG. 3 is a graph showing that sugars only have limited effects on persister formation compared to brominated furanones;

FIG. 4 is a graph of the viability and persistence of P. aeruginosa PAO1 persister cells after incubation for 2 h in 0.85% NaCl buffer in the presence of BF8 at different concentrations;

FIG. 5 is a graph of viability and persistence of P. aeruginosa PAO1 persister cells after incubation for 2 h in 0.85% NaCl buffer in the presence of non-brominated furanones at different concentrations;

FIG. 6 is a graph showing BF9 can reduce antibiotic tolerance of P. aeruginosa PAO1 persisters;

FIG. 7 is a graph showing BF10 can reduce antibiotic tolerance of P. aeruginosa PAO1 persisters;

FIG. 8 is a graph showing BF11 can reduce antibiotic tolerance of P. aeruginosa PAO1 persisters; and

FIG. 9 is a graph showing BF14 can reduce antibiotic tolerance of P. aeruginosa PAO1 persisters.

### DETAILED DESCRIPTION OF THE INVENTION

Referring now to the drawings, wherein like reference numerals refer to like parts throughout, following is an explanation of the materials and method of the present invention.

Bacterial Strain and Growth Medium.

Pseudomonas aeruginosa PAO1 was routinely grown in Luria-Bertani (LB) medium. To isolate persister cells, an overnight culture was incubated for 18 h at 37° C. with shaking at 200 rpm, washed twice with 0.85% NaCl buffer, resuspended in the same buffer and then treated with 200 μg/mL Cip for 3.5 h to lyse the regular cells. The remaining persister cells were then harvested by centrifugation at 13,200 rpm for 3 min at room temperature and then treated with or without furanones as described below.

Furnaone Synthesis.

Furanones BF1, BF8, BF9, BF10, BF11, BF12 and BF14 were synthesized as described previously. The furanones were dissolved in absolute ethanol at 60 mg/mL and stored at 4° C. until use. The non-brominated furanones NF1 and NF2 were obtained from commercial sources.

Quorum Sensing Assay

To understand the effects of BFs on quorum sensing, the reporter strain Vibrio harveyi BB886 was used to monitor the quorum sensing based on homoserine lactones. Briefly, an overnight culture of this strain was diluted by 1:5000 in autoinducer bioassay medium with BFs supplemented at different concentrations. The bioluminescence was measured at 5 h after inoculation.

Effect of Furanone BF8 on Persister Formation.

A P. aerugionsa PAO1 overnight culture was sub-cultured into 6 tubes with 5 mL fresh LB medium in each with the optical density at 600 nm (OD600) adjusted to 0.05. Each tube contained different concentrations of BF8 (0, 5, 10, 30, 50 and 100 μg/mL) diluted from a 60 mg/mL stock solution. The amount of ethanol was adjusted to be the same for all samples to eliminate solvent effects. After 5 h of incubation, 2 mL sample from each tube was washed and plated on LB agar plates for counting colony forming units (“CFU”); while 3 mL of sample was added with 200 μg/mL Cip and incubated for 3.5 h to count the numbers of cells that remained as persisters.

Effects of Sugars

To compare with the effects of sugars known to promote antibiotic uptake by persisters, the above experiment was repeated using 10 mM glucose and mannitol instead of BF8.

Effect of BF8 on the Viability of P. aeruginosa PAO1 Persister Cells.

To obtain persisters, 3 mL of overnight culture of P. aeruginosa PAO1 was incubated for 3.5 h with 200 μg/mL Cip to kill all regular cells. The remaining persisters were washed twice with and resuspended in 0.85% NaCl buffer with 1:50 dilution. Samples were taken immediately to count the CFUs of persisters. Then, each 5 mL of 0.85% NaCl buffer containing persister cells was treated for 3.5 h with 0, 0.1, 0.5, 1 and 2 μg/mL of BF8. The amount of ethanol was adjusted to be the same for all samples. CFUs of treated persister cells were counted to understand if BF8 has any effects on the viability of persister cells.

Effects on the Antibiotic Susceptibility of P. aeruginosa PAO1 Persisters.

The persister cells treated with BF8 as described above were then treated again with 200 μg/mL Cip to understand if any persister cells restored susceptibility to Cip. The CFUs were counted as described above.

DNA Microarray Analysis.

Persister cells were harvested from a 100 mL 18-h culture of P. aeruginosa PAO1. The isolated persister cells were resuspended in 0.85% NaCl buffer supplemented with 1 μg/mL (3.7 μM) BF8 or with the same amount of ethanol (4.17 μL to eliminate the solvent effect as the control). After incubation at 37° C. for 1 h, cells were collected by centrifugation at 10,000 rpm for 5 minutes at 4° C., transferred to 2 mL pre-cooled microcentrifuge tubes and frozen in an ethanol-dry ice bath. The cell pellets were stored at −80° C. until RNA isolation.

The RNeasy Mini Kit (Qiagen) was used to isolate total RNA. First, the cells were lysed by beating at 4,800 oscillations/min using a mini-bead beater (Biospec Products Inc., Bartlesville, Okla.) with 0.5-mm glass beads, 900 uL RLT buffer and 1% 2-Mercaptoethanol. The total RNA was extracted following the manufacture's protocol with additional wash with RW1 buffer, RPE buffer and DNase treatment (RNase-Free DNase Set, Qiagen). The RNA samples were sent to the DNA microarray Facilities at SUNY Upstate Medical University for microarray hybridization. A total of three biological replicates were tested to identify consistently induced/repressed genes.

RNA Slot Blotting.

A total of five genes were tested to confirm the microarray results including PA3523, PA2931, PA0182, PA4167 and PA4943. Primers were designed to include only small inner regions of these genes, around 400 bp on average (see Table 1). Hybridization probes were labeled by using DIG-dUTP in PCR reactions according to manufacturer instructions (PCR DIG Probe Synthesis Kit, Cat No. 16363090, Roche, Mannheim, Germany). RNA samples were loaded and fixed on positively charged nylon membrane (Manhaim Boehringer, Cat. No. 1209272). RNA samples and probes were hybridized on membrane by using Roche DIG Easy Hyb Buffer (Cat. No. 1603558). Then Roche DIG Wash and Block Buffer Set (Cat. 1585762) was used to prepare membranes for imaging.

TABLE 1
Genes and primers to synthesize probe for blotting.

| Gene | Forward Primer | Reverse Primer |
|---|---|---|
| PA4943 | GAAACGGTGGCATTCGTC | GTTTCCAGCTGGGTCTCG |
| PA3523 | CCAGCAACTGTTCCTCATCG | CAGGTAGGTGCGCTCGTC |
| PA2931 | CGAGGCGATGGAAATCAG | GCATAGAAGGTCGCCAACTC |
| PA0182 | CGACATCCTGGTCAACAATG | GGTGATGTAGGCCGCTTC |
| PA4167 | GCAGATCTACGGCAACGAG | GCAAGTAAGGGCTGAGTTCG |

Results

The brominated furanones referenced in the present application and used to obtain the results discussed herein include, but are not limited to, the brominated furanones found in Table 2.

TABLE 2
Representative Brominated Furanone Chemical Structures

| Name | Chemical Structure |
|---|---|
| BF1 | 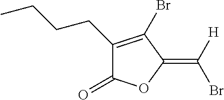 |
| BF2 | 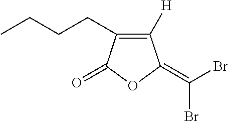 |
| BF3 | 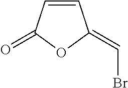 |
| BF4 | 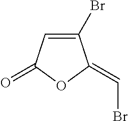 |
| BF5 | 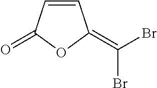 |
| BF6 | 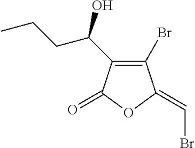 |
| BF7 | 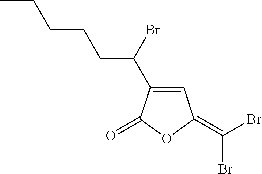 |
| BF8 | 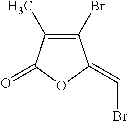 |
| BF9 | 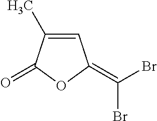 |
| BF10 | 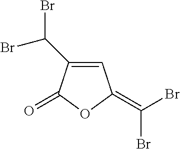 |
| BF11 | 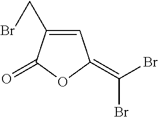 |
| BF12 | 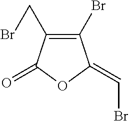 |
| BF13 | 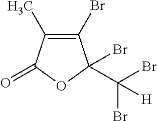 |
| BF14 | 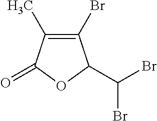 |
| BF15 | 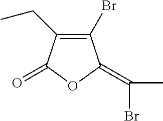 |
| BF16 | 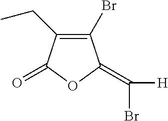 |
| BF17 | 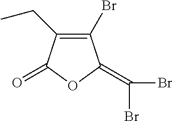 |
| BF18 | 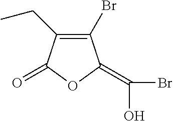 |
| BF19 | 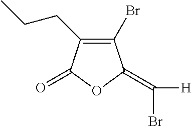 |
| BF20 | 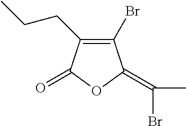 |
| BF21 | 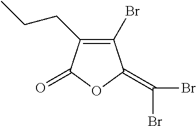 |
| BF22 | 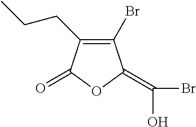 |
| BF23 | 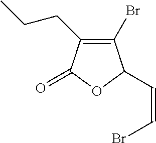 |
| BF24 | 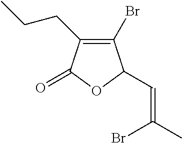 |
| BF25 | 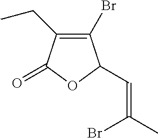 |
| BF26 | 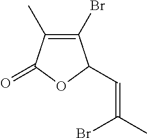 |
| BF27 | 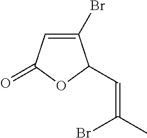 |

Quorum Sensing Inhibition

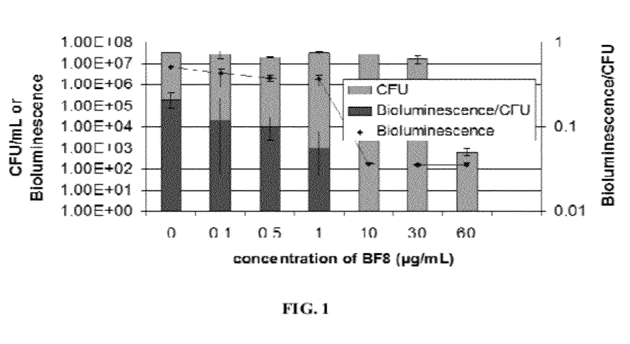

Brominated furanones are inhibitors of bacterial quorum sensing. For example, as shown in FIG. 1, BF8 at 10 μg/mL completely eliminated the response of the reporter strain (V. harveyi BB886) to homoserine lactones.

Persister Control

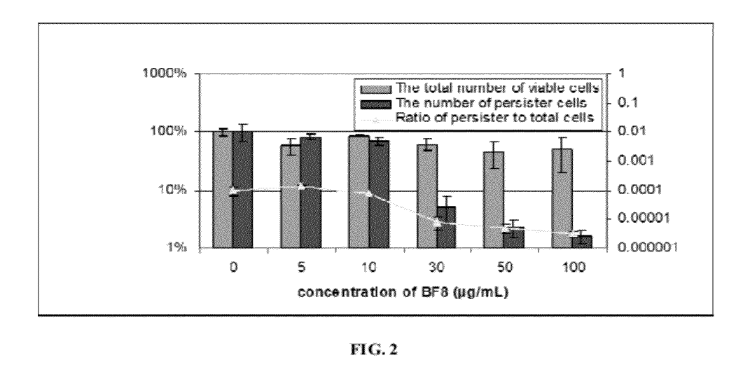

BF8 at 100 μg/mL was found to reduce persister formation when this compound was added in the subcultures of P. aeruginosa PAO1. As shown in FIG. 2, BF8 reduced the number of persister cells formed during the 5 h of incubation by up to 100 times compared to the furanone-free control.

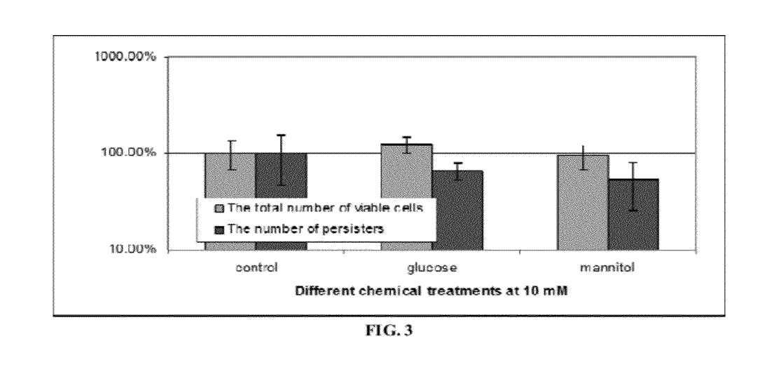

Recently, it was reported that some sugars, such as mannitol, glucose, fructose and pyruvate, can generate proton-motive force and promote the uptake of aminoglycosides by persister cells. To compare our BFs with these sugar molecules for their activities in persister control, the experiment described in FIG. 1 was repeated using glucose and mannitol instead of BF8. It was found that with these sugars at mM level, more than 50% of persisters retained the antibiotic tolerance (FIG. 3), while only 1.6% were persistent after the same treatment with 100 μg/mL BF8. Thus, the brominated furanones are significantly more effective in reducing persister formation.

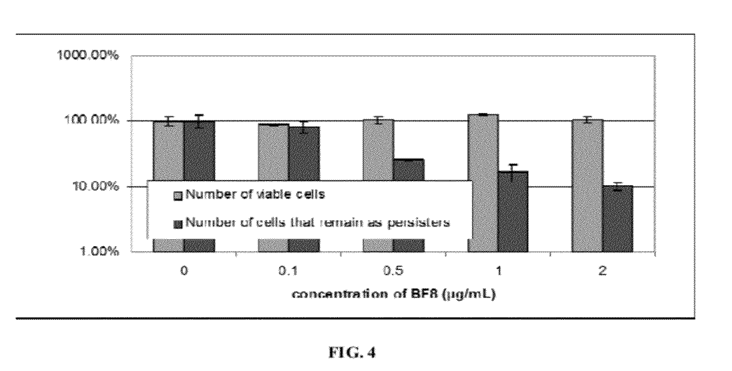

When tested on isolated persisters, furanone BF8 up to 2 μg/mL did not exhibit any significant killing effects on the persister cells of PAO1. At 2 μg/mL; however, BF8 at concentration of 0.5, 1 and 2 μg/mL rendered the PAO1 persisters more susceptible to Cip. For example, at 2 μg/mL around 90% of persister cells were rendered sensitive to Cip. Since BF8 had no effects on the viability of persister cells, these results suggest that BF8 restored their susceptibility to antibiotics (FIG. 4).

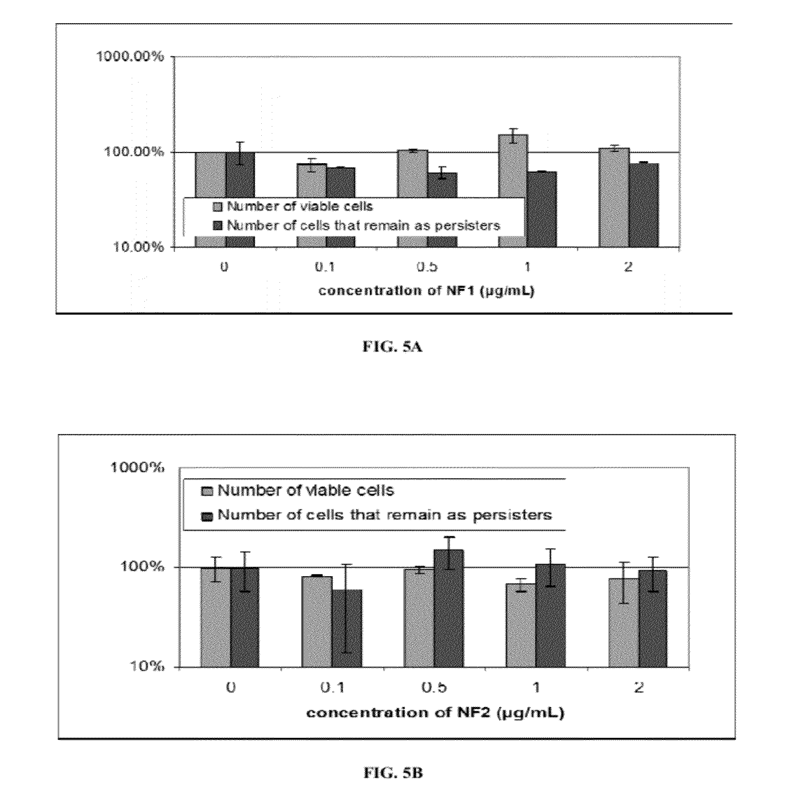

The bromine atoms in BF molecules were found to be essential since the non-brominated furanones (NF1 and NF2) were not effective (FIG. 5 A&B).

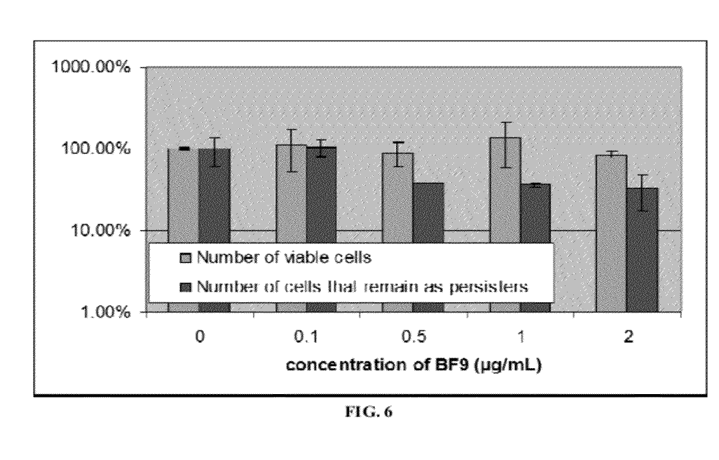

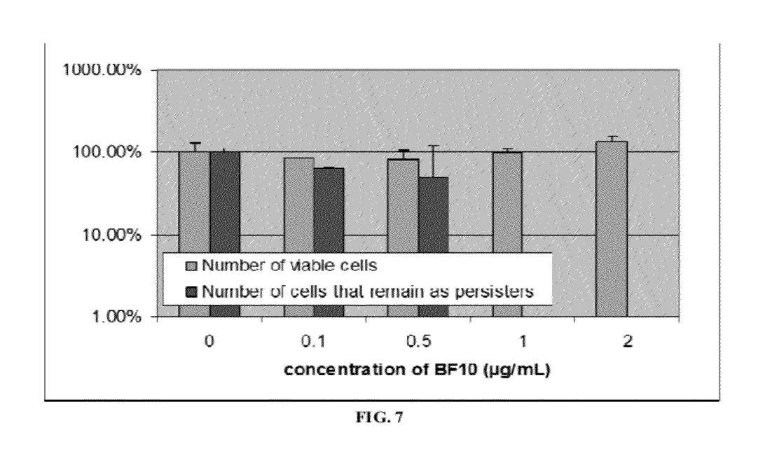

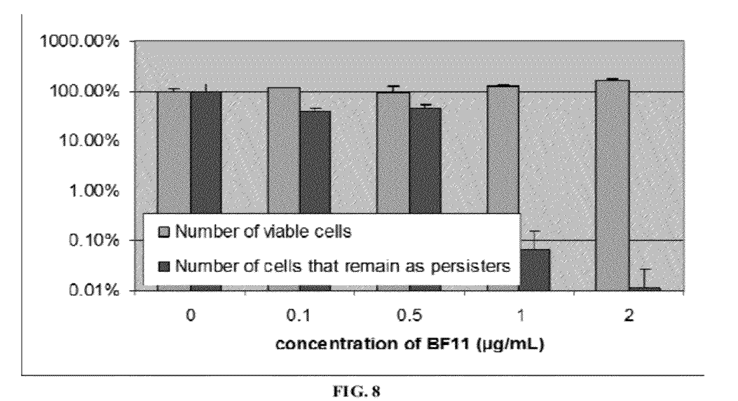

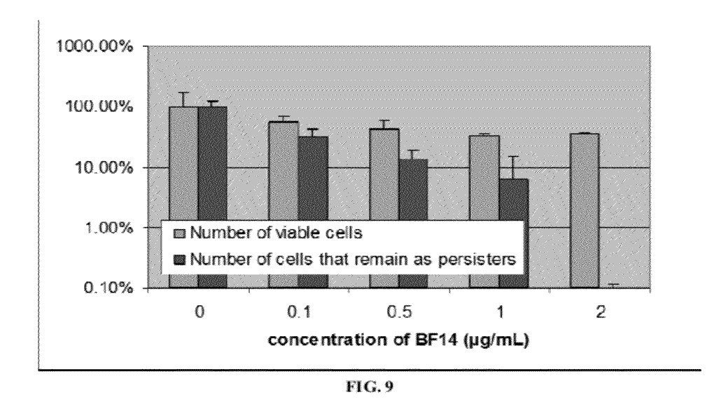

Similar to BF8, several other brominated furanones were also found to enhance the antibiotic susceptibility. As shown in FIGS. 6 to 9, BF9, BF10, BF11 and BF14 were all found to revert antibiotic tolerance in a dose dependent manner.

To better understand the mechanism of persister control by BFs, the gene expression of P. aeruginosa PA01 persister cells treated with and without 1 μg/mL BF8 for 1 h was studied using DNA microarrays. A total of 28 genes were consistently induced by BF8 in all three biological replicates. The induced genes have functions of oxidoreductase synthesis, transport, transcription, and unknown functions (Table 3). The DNA microarray data were verified with RNA slot blotting of four representative genes. Thus, BF8 appeared to influence the membrane potential and function. The fact the BF8 can potentiate Cip, a fluoroquinolone antibiotic, suggests that BFs can also enter the cells and interrupt DNA replication.

TABLE 3
List of genes in P. aerugionsa PAO1 persister cells induced by BF8.

| | Expression | |
|---|---|---|
| Induced Gene | Ratio | Functions |
| PA4167 | 510.6 | 2,5-diketo-D-gluconate reductase B |
| PA1334 | 227.6 | oxidoreductase |
| PA4173 | 36.7 | hypothetical protein |
| PA0182 | 97.1 | 3-ketoacyl-(acyl-carrier-protein) reductase |
| PA2932_morB | 64.9 | morphinone reductase |
| PA0741 | 16.5 | hypothetical protein |
| PA1210 | 21 | hypothetical protein |
| PA3240 | 14.4 | hypothetical protein |
| PA3523 | 9.6 | Resistance-Nodulation-Cell Division |
| | | (RND) efflux membrane fusion |
| | | protein precursor |
| PA2535 | 9 | oxidoreductase |
| PA2575 | 9.1 | hypothetical protein |
| PA2931 | 11 | CifR |
| PA0565 | 12.8 | hypothetical protein |
| PA2580 | 8.3 | hypothetical protein |
| PA2610 | 7.2 | hypothetical protein |
| PA2839 | 11 | hypothetical protein |
| PA0422 | 4.2 | hypothetical protein |
| PA3223_acpD | 4.8 | AzoR3, azoreductase 3 |
| PA1374 | 3.4 | hypothetical protein |
| PA3920 | 3.9 | metal transporting P-type ATPase |
| PA4878 | 4.2 | transcriptional regulator |
| PA1285 | 4.4 | transcriptional regulator |
| PA1470 | 4.1 | short chain dehydrogenase |
| PA3133 | 3.5 | transcriptional regulator |
| PA2196 | 4.8 | transcriptional regulator |
| PA2378 | 3.1 | aldehyde dehydrogenase |
| PA2691 | 3.7 | hypothetical protein |
| PA1127 | 3.4 | oxidoreductase |

The extremely enhanced tolerance to antibiotics by persister cells presents a serious challenge to antibiotic therapies. To solve this problem, it is important to develop new methods to effectively kill persister cells or to restore their susceptibility to antibiotics. The activities of BF8 represent the first non-metabolite small molecule to revert antibiotic tolerance of persister cells. Since BF8 is a known inhibitor of quorum sensing and quorum sensing has been known to promote persister formation in P. aeruginosa PAO1, the activity of BF8 on persister cells may be partially through quorum sensing inhibition; while the effects on membrane genes, etc., indicate that BFs may have other targets for persister control and can be effective against a broad spectrum of bacterial species.

Compared to sugars, BFs have unique advantages in persister control. The BF compounds are not metabolites like sugars and do not stimulate bacterial growth even at high concentrations. In fact, high concentrations of BFs are cidal to bacterial cells. Thus, it is easier to apply in vivo. In addition, the BF molecules can enhance the susceptibility of persister cells to fluoroquinolones antibiotics, which cannot be obtained with sugar treatment. Since such antibiotics attack DNA replication, the activity of BFs suggests that these compounds work through a different mechanism of membrane potentiating by sugars.

The present invention is applicable to the treatment of chronic wounds, chronic sinusitis, implanted device associated infections, middle ear infections, tuberculosis and the like. The present invention may also be employed for decontamination of items such as medical devices and for treatment plant diseases caused by bacteria.

Although the present invention has been described in connection with a preferred embodiment, it should be understood that modifications, alterations, and additions can be made to the invention without departing from the scope of the invention as defined by the claims.

What is claimed is:

1. A method for reverting the antibiotic tolerance of a bacterial persister cell, the method comprising the step of contacting the bacterial persister cell with a brominated furanone.

2. The method of claim 1, wherein the step of contacting the bacterial persister cell with said brominated furanone does not stimulate full growth of the cell.

3. The method of claim 1, wherein said brominated furanone activates a transport activity of an outer membrane of said bacterial persister cell.

4. The method of claim 1, wherein said brominated furanone activates transcription of at least gene in said persister cell.

5. The method of claim 1, wherein brominated furanone is a quorum sensing inhibitor.

6. The method of claim 1, wherein said brominated furanone comprises one or more compounds selected from Table 2.

7. The method of claim 1, wherein said bacterial persister cell is a microorganism selected from the group consisting of Pseudomonas aeruginosa, Burkholderia cepacia, Salmonella typhimurium, Vibrio fisheri, V. harveyi, V. cholera, Aeromonas hydrophila, Serratia liquefaciens, Erwinia carotovora, Agrobacterium tumefaciens, and mixtures thereof.

8. The method of claim 1, wherein the bacterial persister cell is in a biofilm.

9. A method to inhibit or eliminate a bacterial infection comprising at least one bacterial persister cell, the method comprising the steps of:
administering a brominated furanone to said bacterial infection, wherein said brominated furanone reverts the antibiotic tolerance of said at least one bacterial persister cell; and
administering at least one antimicrobial agent to said bacterial infection.

10. The method of claim 9, wherein the step of administering a brominated furanone to said bacterial infection comprises contacting said at least one bacterial persister cell with said brominated furanone.

11. The method of claim 9, wherein the step of administering a brominated furanone to said at least one bacterial persister cell does not stimulate full growth of the cell.

12. The method of claim 9, wherein the step of administering a brominated furanone to said at least one bacterial persister cell activates a transport activity of a membrane of said bacterial persister cell.

13. The method of claim 9, wherein the step of administering a brominated furanone to said at least one bacterial persister cell activates transcription of at least gene in said persister cell.

14. The method of claim 9, wherein said brominated furanone comprises one or more compounds selected from Table 2.

15. The method of claim 9, wherein said at least one bacterial persister cell is a microorganism selected from the group consisting of Pseudomonas aeruginosa, Burkholderia cepacia, Salmonella typhimurium, Vibrio fisheri, V. harveyi, V. cholera, Aeromonas hydrophila, Serratia liquefaciens, Erwinia carotovora, and mixtures thereof.

16. The method of claim 9, wherein said antimicrobial agent is selected from the group consisting of a β-lactam, an aminoglacoside, a quinolone, a tetracycline, a cephalosporin, and mixtures thereof.

17. The method of claim 9, wherein said bacterial infection is present in an animal or a plant.

18. A method of preventing a bacterial infection from developing on a surface, comprising the steps of:
administering a brominated furanone to said surface; and
administering at least one antimicrobial agent to said surface.

19. The method of claim 18, wherein said surface comprises a surface of an implantable device.

20. The method of claim 18, wherein said surface comprises a surface of a wound dressing.
